(19) United States
(12) Patent Application Publication
(10) Pub. No.: US 2012/0255546 A1
(43) Pub. Date: Oct. 11, 2012

(54) PORTABLE VAPORIZER

(75) Inventors: David Robert Goetz, Los Angeles, CA (US); William Tan, Los Angeles, CA (US); Alfred Scaglione, Los Angeles, CA (US)

(73) Assignee: VISIONARY ROAD, Los Angeles, CA (US)

(21) Appl. No.: 13/444,818

(22) Filed: Apr. 11, 2012

Related U.S. Application Data
(60) Provisional application No. 61/474,081, filed on Apr. 11, 2011.

(51) Int. Cl. A61M 15/06

(52) U.S. Cl. 128/202.21

(57) ABSTRACT

A portable vaporizer includes a main body, a removable herbal attachment configured to vaporize and herb, a removable cigarette attachment configured to vaporize the contents of a cigarette, wherein the main body is configured to attach interchangeably to the removable herbal attachment and the removable cigarette attachment.

### CROSS-REFERENCE TO RELATED APPLICATION(S)

The present application for patent claims priority to U.S. Provisional Application No. 61/474,081, entitled “Portable Vaporizer”, filed Apr. 11, 2011, of which is assigned to the assignee hereof and hereby expressly incorporated by reference here.

### BACKGROUND

1. Field

The present disclosure relates generally to a vaporizer, and more particularly, to a portable vaporizer having a main body that is configured to interchangeably connect with multiple removable attachments for vaporizing different materials.

2. Background

A vaporizer is a device or system for extracting and delivering the active ingredients of plant material for inhalation. The plant material may contain tobacco, herbs, or blends. Vaporization is an alternative to smoking. Rather than burning the herb, which can cause irritation from toxic and carcinogenic by-products, a vaporizer heats the material so that its active compounds boil off into a vapor. Vaporization requires a temperature just below burning in order to receive elements from the herb without producing carbon dioxide and other noxious gases. Vaporization provides a more pure sample of the herb. Further, vaporization eliminates the presence and taste of smoke, instead, producing a vapor having essentially no particulate matter or tar. The elimination of smoke lowers the concentrations of noxious gases such as carbon monoxide and reduces the harmful effects of smoking and second hand smoke. Additionally, vaporization offers the advantage of a rapid onset of pharmacological effect.

Vaporizers are often large and bulky. Although portable, hand-held vaporizers have been manufactured, they typically use a butane flame heater to vaporize an herb within a steel container. Among other drawbacks, the heated steel produces an undesirable metallic smell along with the vapor. Conventional hand-held vaporizers have limited types of materials that they are able to vaporize.

### SUMMARY

Aspects presented herein overcome these disadvantages by providing an interchangeable portable vaporizer having a main body that attaches to various attachment pieces for vaporizing different types of materials. For example, one attachment piece may include a bowl shaped opening for receiving loose herbs. Another may include a cigarette holder that holds a standard cigarette and releases the tobacco within the cigarette to be vaporized.

In an aspect of the disclosure, a portable vaporizer includes a main body, a removable herbal attachment configured to vaporize and herb, a removable cigarette attachment configured to vaporize the contents of a cigarette, wherein the main body is configured to attach interchangeably to the removable herbal attachment and the removable cigarette attachment.

The portable vaporizer may include a battery to power a heating element, such as a ceramic heating element, in the removable attachments. The heating element may be separated from the material to be vaporized by a glass layer. This causes vaporization of the material via thermal radiation rather than via convection.

The portable vaporizer may include a damper and an articulated cap that enables the damper to be inserted vertically into the bowl when closing the cap. The damper may be configured to compress and mix the material within the bowl.

Additional advantages and novel features of aspects will be set forth in part in the description that follows, and in part will become more apparent to those skilled in the art upon examination of the following or upon learning by practice thereof.

### BRIEF DESCRIPTION OF THE DRAWINGS

So that the manner in which the above-recited features of the disclosure set forth herein can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects.

FIG. 1 is a first perspective view of a portable vaporizer having an herbal attachment.

FIG. 2 is a second perspective view of the portable vaporizer of FIG. 1.

FIG. 3 is a transparent view of an internal portion of the herbal attachment of FIG. 1.

FIG. 4 is a bottom perspective view of the portable vaporizer FIG. 1.

FIG. 5 is an exploded view of a removable pipe being stored with the portable vaporizer of FIG. 1.

FIG. 6 is an exploded view of the herbal attachment and the portable vaporizer of FIG. 1.

FIG. 7 is a top perspective view of a main body portion of the portable vaporizer of FIG. 1.

FIG. 8 is a top perspective view of the herbal attachment of the portable vaporizer of FIG. 1.

FIG. 9 is a side view of the herbal attachment of the portable vaporizer of FIG. 1.

FIG. 10 is a perspective view of a portable vaporizer having a cigarette attachment configured in accordance with another embodiment of the present invention.

FIG. 11 is an exploded view of the cigarette attachment of FIG. 10.

FIG. 12 is a bottom perspective view of a cigarette holder for the cigarette attachment of FIG. 10.

FIG. 13 is a bottom perspective view of the cigarette holder of FIG. 12 with a cigarette inserted therein.

FIG. 14 is an exploded view of the cigarette attachment of FIG. 10 with the cigarette inserted therein.

FIG. 15 is a transparent view of an internal portion of the cigarette attachment of FIG. 10.

FIGS. 16a, 16b is a perspective view of an integrated portable vaporizer configured in accordance with another embodiment of the present invention.

FIG. 17 is a close-up perspective view of an articulating hinge for the integrated portable vaporizer of FIG. 16.

FIGS. 18a, 18b is a close-up perspective view of a damper for the integrated portable vaporizer of FIG. 16.

FIGS. 19a, 19b is a close-up perspective cross section view of the damper and heater assembly for the integrated portable vaporizer of FIG. 16.

FIG. 20a, 20b is a close-up perspective view of the damper and heater assembly for the integrated portable vaporizer of FIG. 16.

FIG. 21 is an exploded parts diagram for a cap assembly for the integrated portable vaporizer of FIG. 16.

FIG. 22 is a perspective cross section view of an air intake for the integrated portable vaporizer of FIG. 16.

FIG. 23 is an exploded perspective view of an herb containing and heating assembly for the integrated portable vaporizer of FIG. 16.

FIG. 24a, 24b is a perspective cross section view of the herb containing and heating assembly of FIG. 23.

FIG. 25 a cross section view of the herb containing and heating assembly of FIG. 23.

FIG. 26 a partial cross section view of the herb containing and heating assembly or FIG. 23 as inserted in the integrated portable vaporizer of FIG. 16.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details.

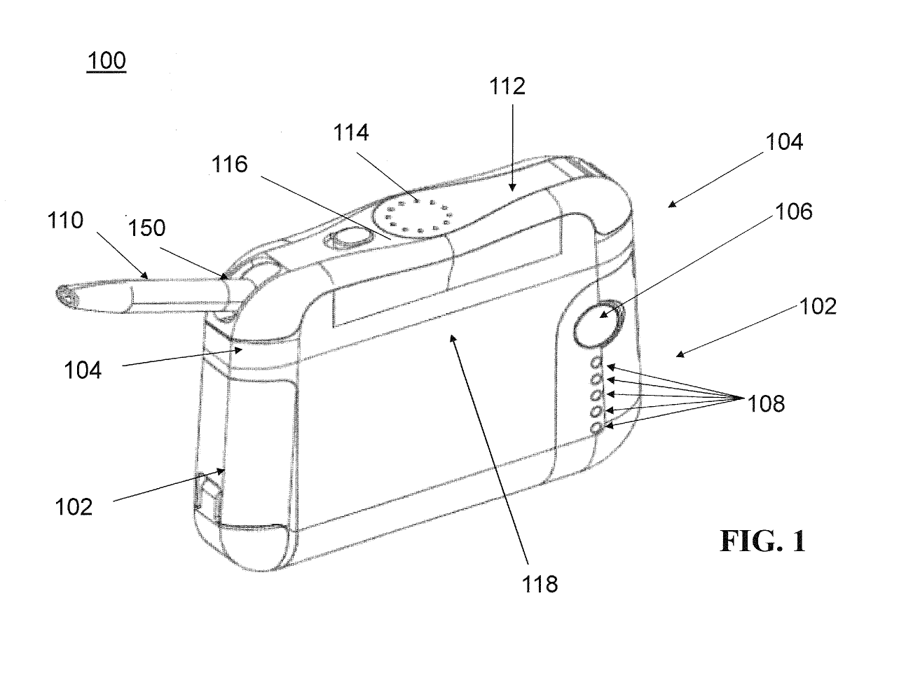

FIG. 1 illustrates example aspects of a first configuration of a portable vaporizer unit 100 having a main body 102 and an herbal attachment piece 104 that is removable from the main body 102. The herbal attachment piece 104 attaches to the main body 102 when in normal use for when the portable vaporizer unit 100 is used to heat herbs. A line separating the main body 102 and the herbal attachment piece 104 is shown at a seam 118. The main body 102 is configured to include a battery for powering a heating element located in the herbal attachment piece 104, as further described herein. A knob 106 enables a user to adjust the temperature of the heating element. The main body 102 may also include indicators 108 that visually show a user, among other things, the temperature level of the heating element, battery power, etc. For example, one of the indicators may be a light, such as a Light Emitting Diode (LED)-type light, that blinks from the time that the heating element begins heating until the heating element reaches the desired temperature. Once the desired temperature is reached, a static light replaces the blinking light. The indicator may use other methods of indicating these features, such as different colored lights indicating that the desired temperature has been reached or a digital display. The herbal attachment piece 104 is configured to receive a removable mouthpiece 110.

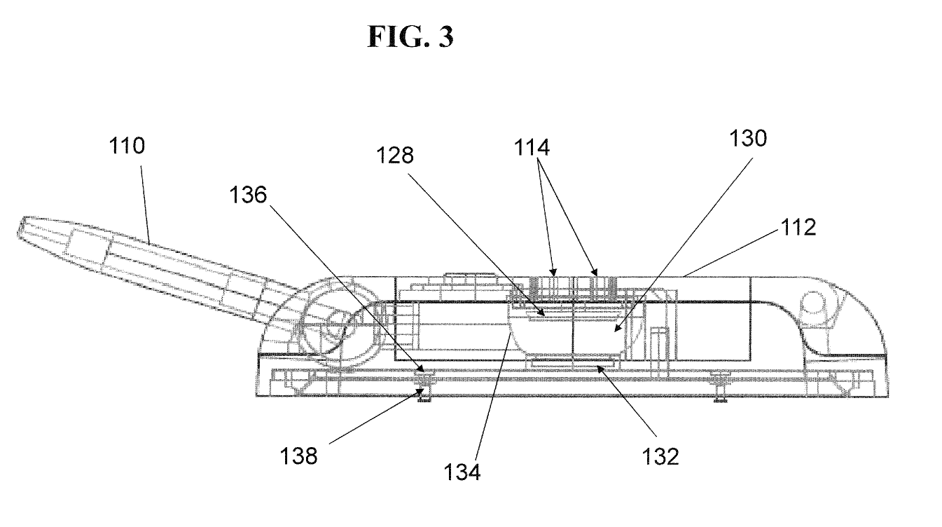

The herbal attachment piece 104 includes a bowl-shaped opening 130, also referred to interchangeably herein as an herb container, for receiving an herb for vaporization, as illustrated in connection with FIGS. 3, 8, and 9. A cover 112 closes over the herb container. The cover 112 includes openings 114 that allow air through the cover 112 to the herb container 130. As illustrated in FIG. 3, the cover 112 further includes a one-way valve 128 located between the cover 112 and the herb container 130. The one-way valve 128 flexes as air is pulled through the mouthpiece, thereby opening and pulling air through the openings 114 in the cover 112 into the herb container 130. Otherwise, the one-way valve 128 remains in a closed position. Further, if air is blown through the mouthpiece into the herb container 130, the valve 128 flexes against the cover 112, maintaining its closed position covering the openings 114. Among other materials, the one-way valve 128 may comprise silicone. The attachment piece also includes a latch 146/148 that closes the cover 112 and a release 116 for releasing the latched cover 112.

The herbal attachment piece 104 further includes a heating element 132. The heating element 132 may be, for example, a ceramic heater. The heating element 132 is located beneath the opening that forms the herb container 130. The herb container 130 is formed in an opening between the cover 112 and a bottom layer 134. The bottom layer 134 may comprise glass.

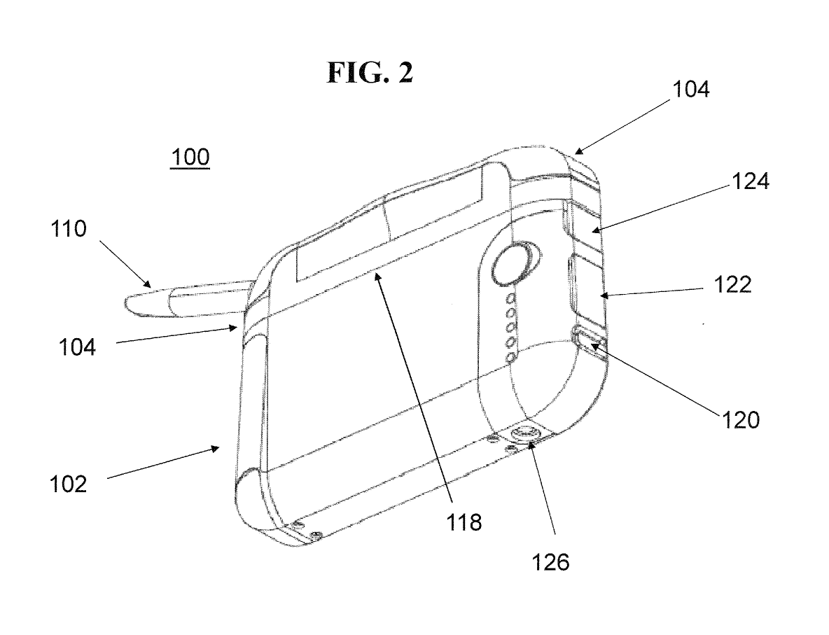

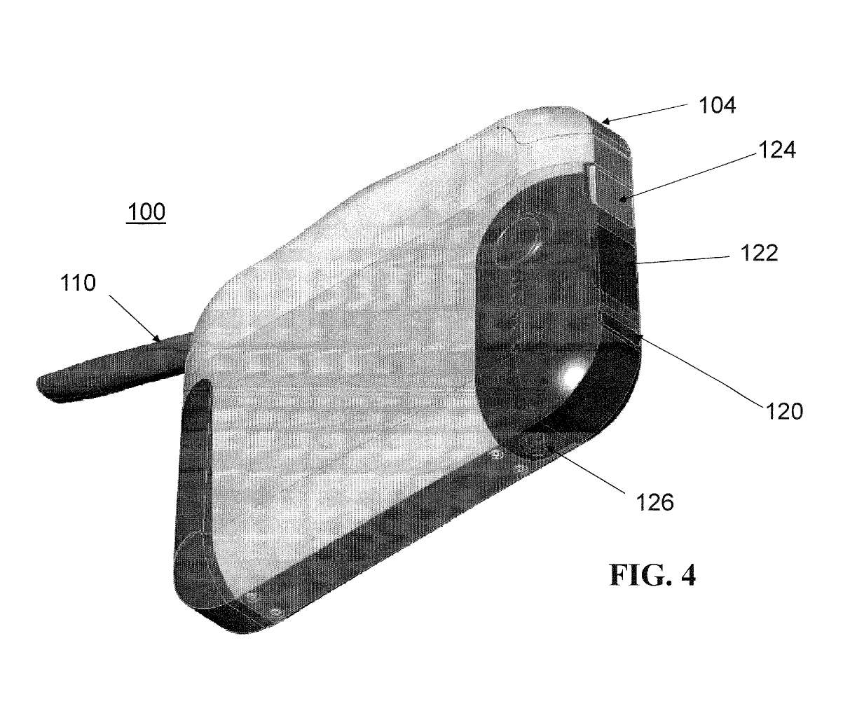

FIGS. 2 and 4 illustrate views of a back and bottom portion of the vaporizer 100. The main body 102 may include a switch 120 for turning the power on and off, a switch 122 for turning the heating element on and off, and a latch 124 that latches and releases the herbal attachment piece 104. Further, as illustrated in FIG. 2, the main body 102 may further include a connection portion 126 that receives a connector for charging the battery within the main body. The connector may be, for example, a DC jack.

FIG. 3 illustrates a view of the internal portions of herbal attachment piece 104. A one-way valve 128 is illustrated between the herb container 130 and the openings 114 in the cover. A glass layer 134 separates the herb container 130 from a heating element 132. FIG. 3 also illustrates components that allow the mouthpiece 110 and the cover 112 to rotate with respect to the main portion of the attachment piece.

Power contacts 136 on the attachment piece 104 are configured to interface or mate with power contacts 138 on the main body 102 in order to power the heating element 132 in the attachment piece.

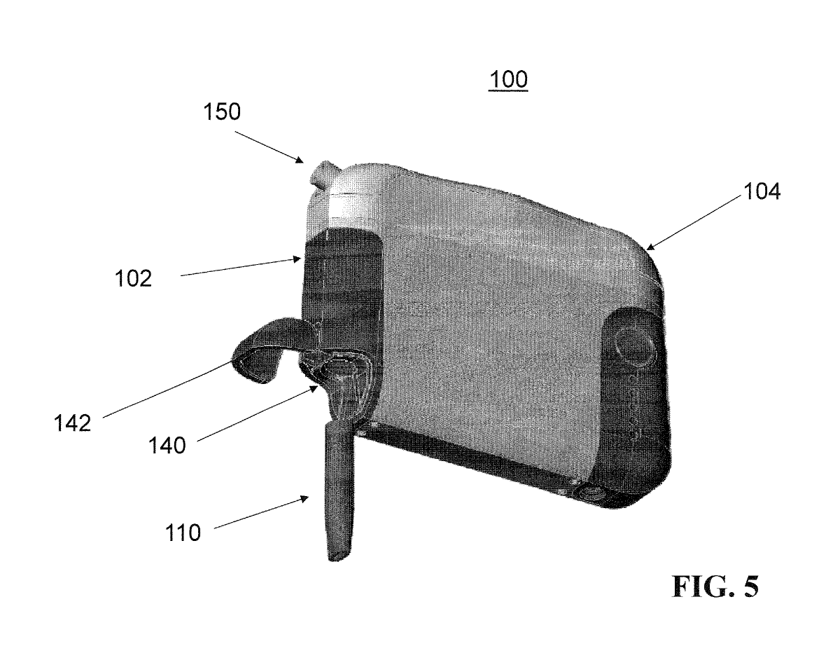

As illustrated in FIG. 5, the main body 102, may include an opening 140 for receiving and storing the mouthpiece 110. A mouthpiece door 142 opens so that the mouthpiece 110 can be inserted into the opening 140 and closes over the mouthpiece to store the mouthpiece inside the main body 102.

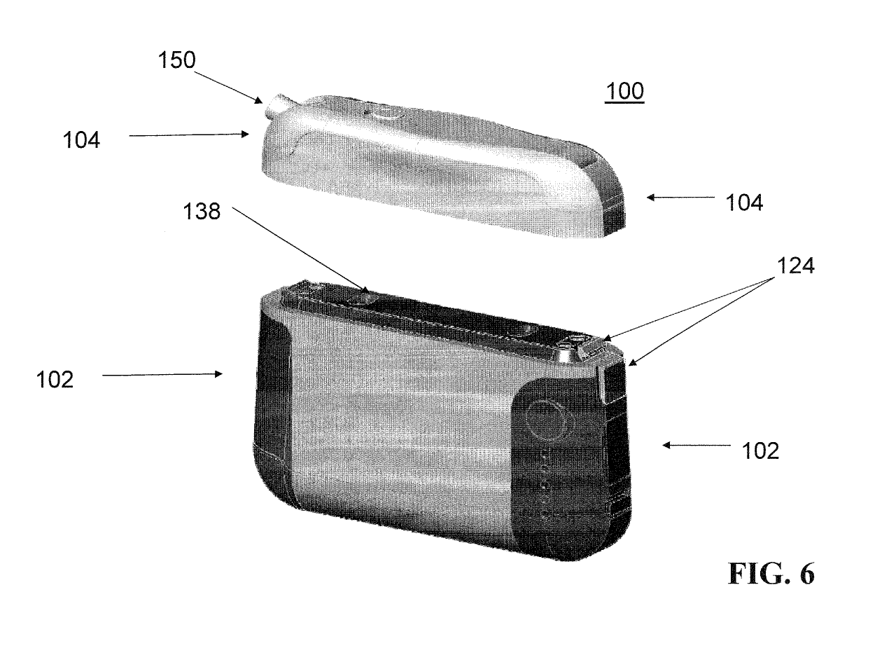

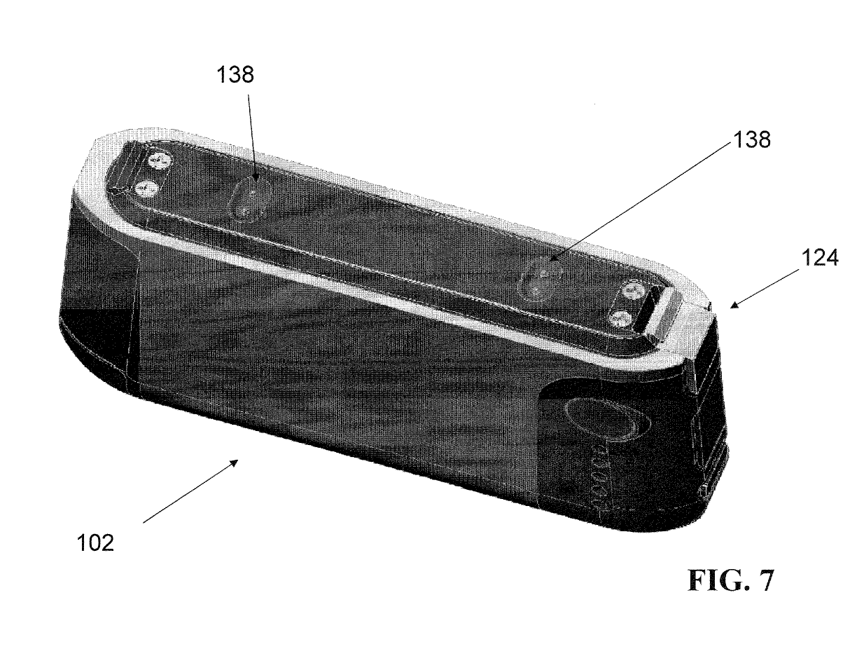

FIG. 6 illustrates the main body 102 and the herbal attachment piece 104 separated from each other. FIG. 7 illustrates a view of the main body 102 separate from the herbal attachment piece 104. The power contacts 138 on the main body 102 are shown. The power contacts 138 may be, for example, spring contacts configured to interface with the power contacts 136 on the attachment piece 102. Also, the latch 124 that holds and releases the herbal attachment piece 104 is illustrated.

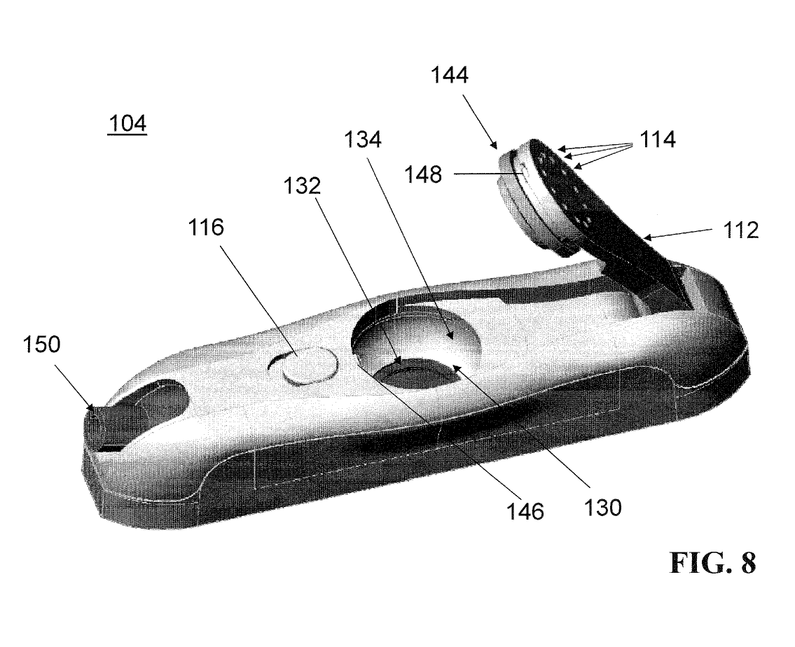

FIG. 8 illustrates the herbal attachment piece 104 with a seal 144 attached to the cover 112 that will form a seal between the cover 112 and the herb container 130 when the cover 112 is closed. The cover lock release 116 includes a latch 146 that latches a portion 148 of the cover 112 to maintain the cover 112 in a closed position. The cover lock 116 slides to release the cover 112.

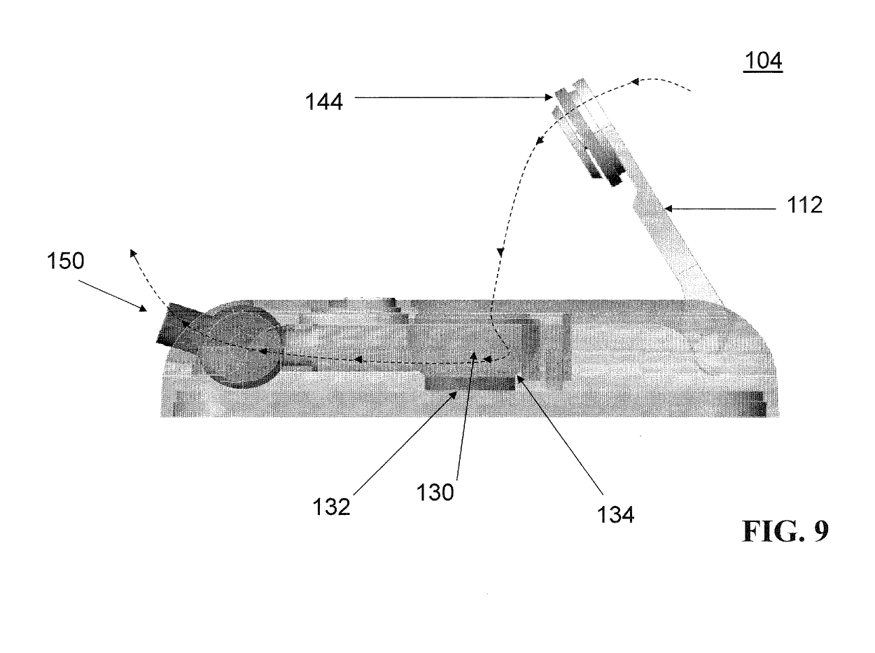

FIG. 9 illustrates an air flow path, as illustrated by a dashed line, through the herbal attachment piece 104 where, once the cover 112 is closed, a user will breathe the vaporized herb by pulling air through the mouthpiece 110. Although the mouthpiece 110 is not illustrated in FIG. 9, the herbal attachment piece 104 includes a mouthpiece connection piece 150 having an opening configured to receive the mouthpiece 110. The mouthpiece connection piece 150 includes an opening that is coupled to the herb container 130 so that vapor from the herb container 130 moves through the mouthpiece connection piece 150 to exit out of the mouthpiece 110. When the user pulls air through the mouthpiece 110, air is drawn through the openings 114 in the cover 112, into the herb container 130. Then, the air flow is mixed with vapor from the herbs being vaporized by the heating element 132 and the vaporized airflow exits through the mouthpiece.

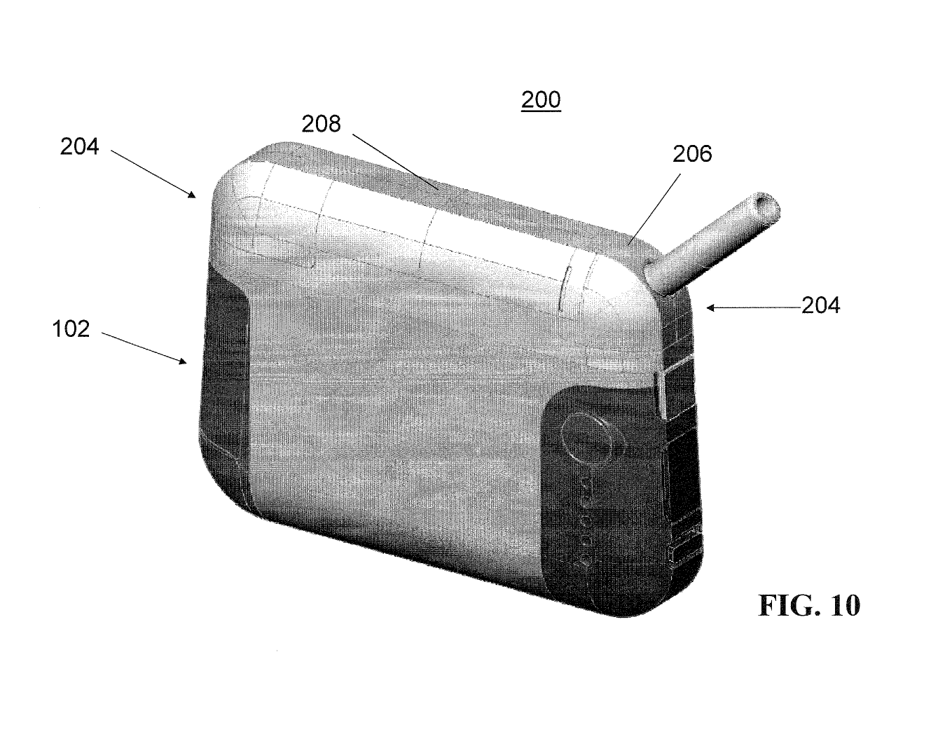

FIGS. 10-15 illustrate a second configuration of a vaporizer 200 having the main body 102 and a cigarette attachment piece 204. The main body 102 can be connected interchangeably with multiple types of attachment pieces. Thus, the same main body 102 that is configured to connect with the herbal attachment piece 104 having the herb container 130 and with cigarette attachment piece 204. The features of the main body 102 illustrated in FIGS. 10-15 are the same as described with the use of the main body 102 in connection with FIGS. 1-9.

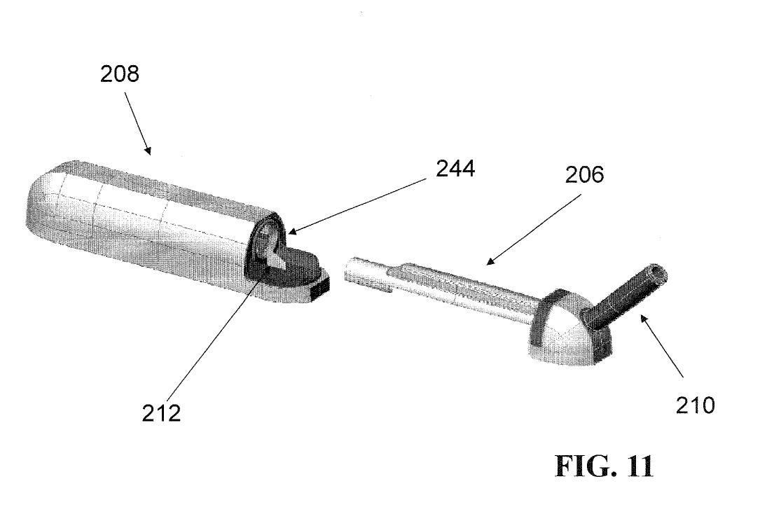

As illustrated in FIGS. 10 and 11, the cigarette attachment piece 204 has a first, heater unit portion 208 and a second, cigarette holder portion 206. The cigarette holder portion 206 is configured to securely hold a cigarette. The heater unit portion 208 has an opening 244 configured to receive the cigarette and cigarette holder portion 208. The first portion 208 and the second portion 206 connect to form the cigarette attachment piece 204. The first, heater unit portion 208 includes a tobacco releasing mechanism. In this example implementation, the tobacco releasing mechanism includes a knife 212 that slices the paper of the cigarette as the second, cigarette holder portion 206 is inserted into and connected to the first heater unit portion 208. By slicing the paper, the tobacco inside the paper is released so that it can be vaporized.

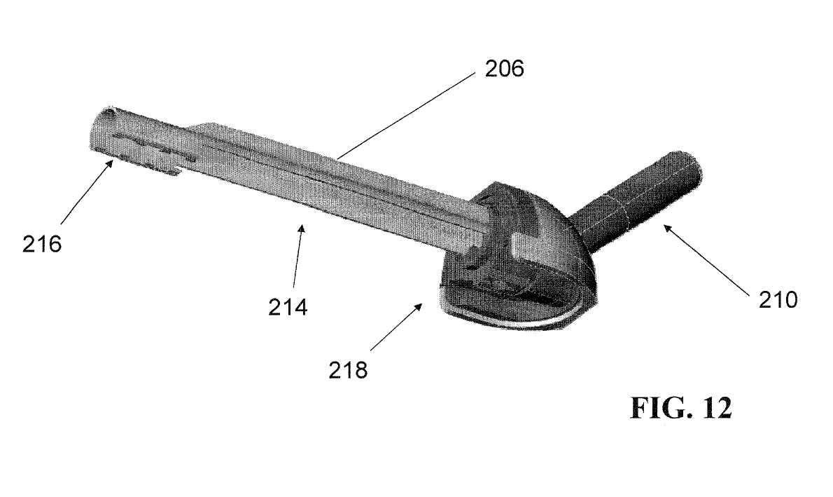

FIG. 12 illustrates the cigarette holder portion 206 shaped to partially surround a cigarette at 214 and having teeth 216 to grip the cigarette. The cigarette holder portion 206 may comprise, for example, stamped metal. The cigarette holder portion 206 is configured to be inserted into the first, heating unit portion 208 and, for example, at 218, to snap together with the heater unit portion 208.

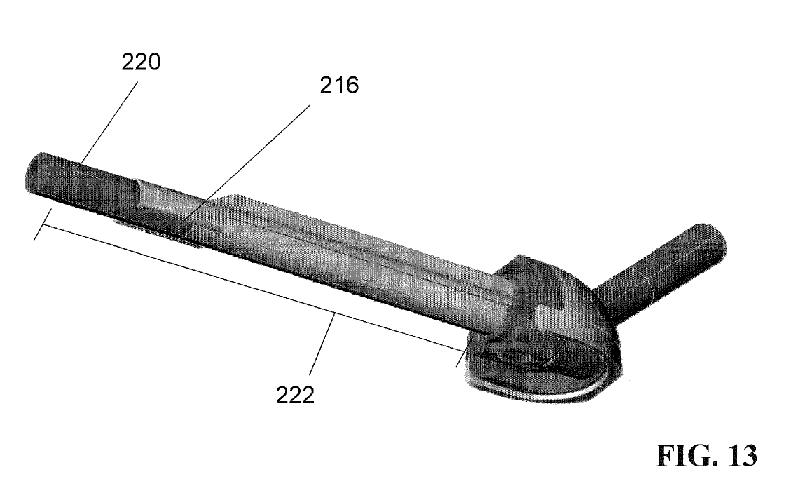

FIG. 13 illustrates the cigarette holder portion 206 holding a cigarette. The teeth 216 grip the filter end 220 of the cigarette, and the cigarette surrounding portion 214 leaves a portion 222 of the cigarette exposed. This exposed portion 222 of the cigarette will be sliced with the knife 212 when the two pieces of the cigarette attachment piece are connected to each other.

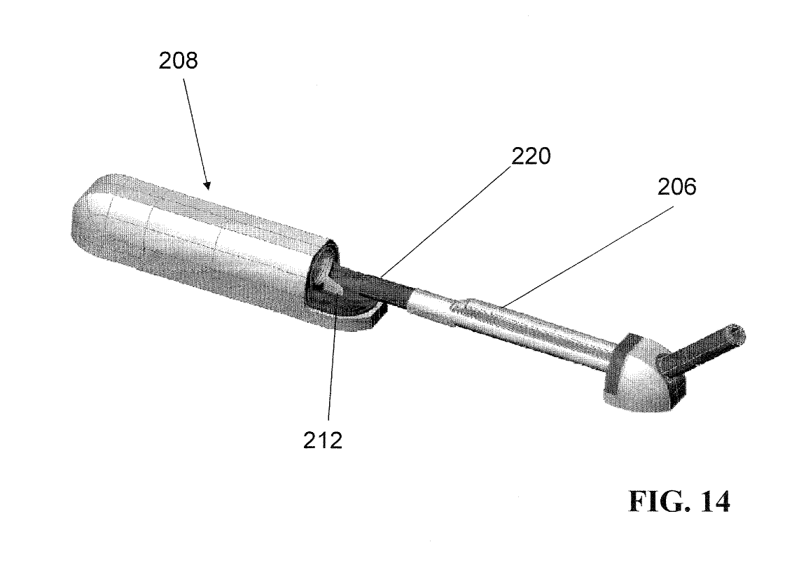

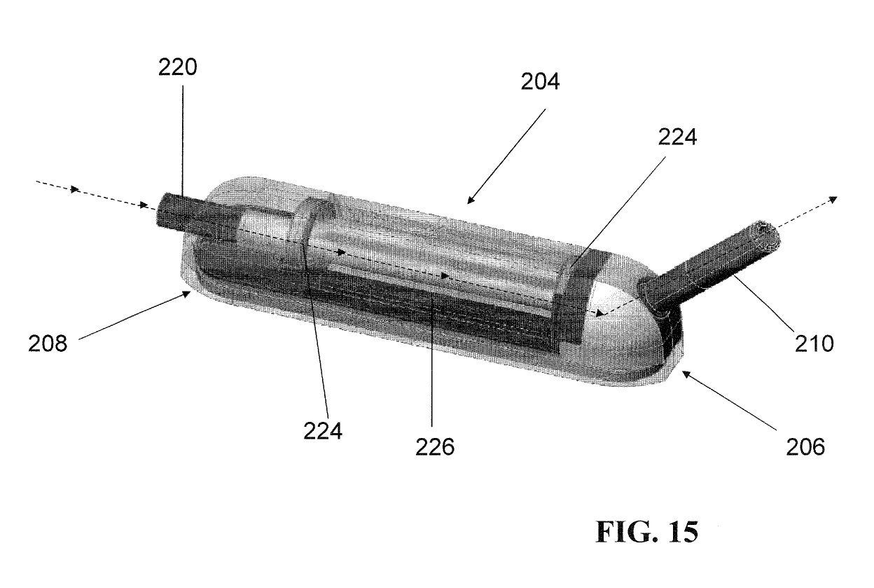

FIG. 14 illustrates the cigarette holder portion 206 being inserted into the heater unit portion 208 while holding a cigarette. FIG. 15 illustrates interior elements of the cigarette attachment 204. The heating unit portion 208 includes a heating element 226. As in the herbal attachment piece 104, the heating element 226 may be a ceramic heater. The heating element in FIG. 15 is rectangular, extending along exposed surface of the cigarette 222 that has been sliced to expose the tobacco inside the cigarette. Similar to the herbal attachment piece 104, the cigarette attachment piece 204 includes a glass layer between the tobacco and the heating element 226. The heater unit portion 208 also includes two gaskets 224 that form a seal around the cigarette holder portion 206 on either side of the heating element 226. The gaskets 224 prevent air and vapor from escaping a vaporization area formed by the gaskets 224, the glass layer between the tobacco and the heating element 226, and the surrounding surface of the heater unit portion 208 and the cigarette holder portion 206. The filter portion of the cigarette may extend into the exterior of the cigarette attachment 204. A dashed line illustrates an air flow during use of the vaporizer 200 with the cigarette attachment 204. When air is pulled through the mouthpiece 210, air is drawn in through the filter end of the cigarette. As the air enters the portion between the gaskets 224, the air is vaporized with tobacco vapor. The vaporized airflow exits through the mouthpiece 210.

By vaporizing the tobacco rather than burning the cigarette, the pharmaceuticals from the tobacco can be received by a user without carbon dioxide and other noxious gases from burning the tobacco.

In one aspect of the disclosed approach, the vaporizer, whether it uses either the example herbal attachment piece 104 or the cigarette attachment piece 204, heats the substance via thermal radiation that passes through the transparency of the glass layer rather than heating the vaporization material by conduction. Glass does not have a high level of conductance, thereby enabling vaporization through thermal radiation.

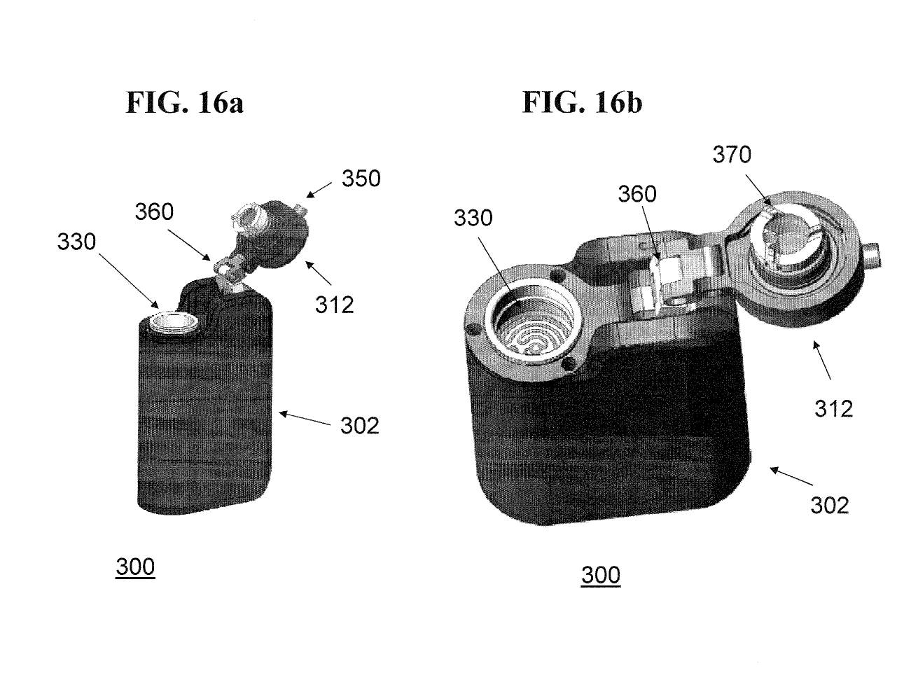

FIGS. 16a and 16b illustrate another portable vaporizer 300 configured in accordance with another embodiment of the present invention having an articulated cap 312. In this example, a heating chamber 330 and the cap 312 may be formed integrally with a main body 302 rather than being removable from the main body 302. The cap 312 includes a mouthpiece connection piece, or pipe mount 350 that is configured to receive a removable a mouthpiece similar to the mouthpiece connection piece 150, as described supra. Although not illustrated, the main body 102 may include a battery or power source, a mouthpiece storage portion, and other features similar to those described for the other vaporizers, supra. The cap 312 attaches to the main body 302 of the vaporizer 300 via an articulated hinge 360. The articulated hinge 360 includes multiple links that enable the cap 312 to be inserted vertically into the opening of the heating chamber 330 when the cap 312 is positioned over the heating chamber 330.

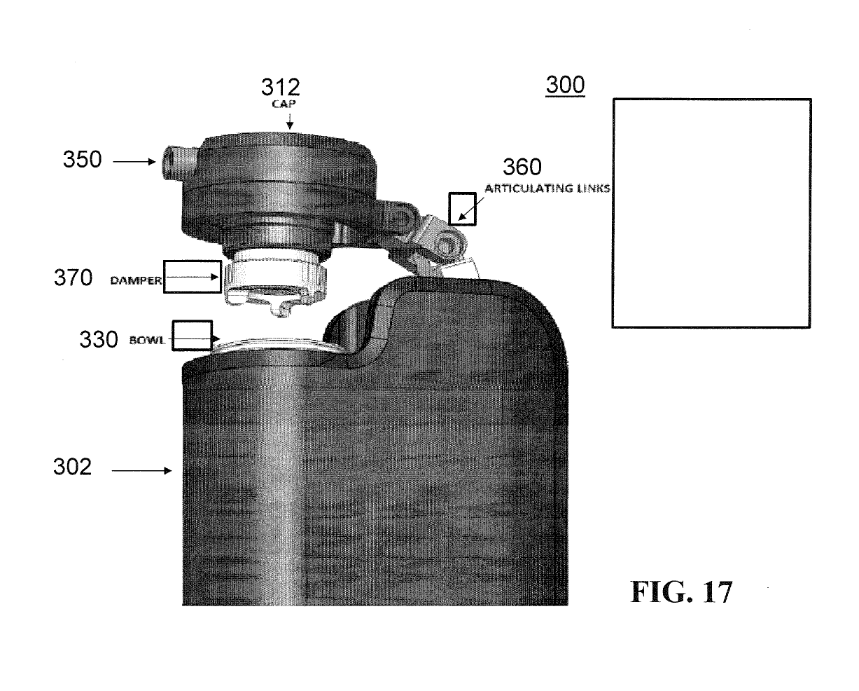

The portable vaporizer 300 includes a damper 370 attached to the cap 312. The damper is configured to compress and agitate the material within the heating chamber 330 so that the material mixes and vaporizes evenly. FIG. 17 illustrates the multiple links within the articulated hinge 360. The damper 370 extends from the cap 312. The articulated hinge 360 enables the damper 370 to be inserted vertically into the opening of the heating chamber 330.

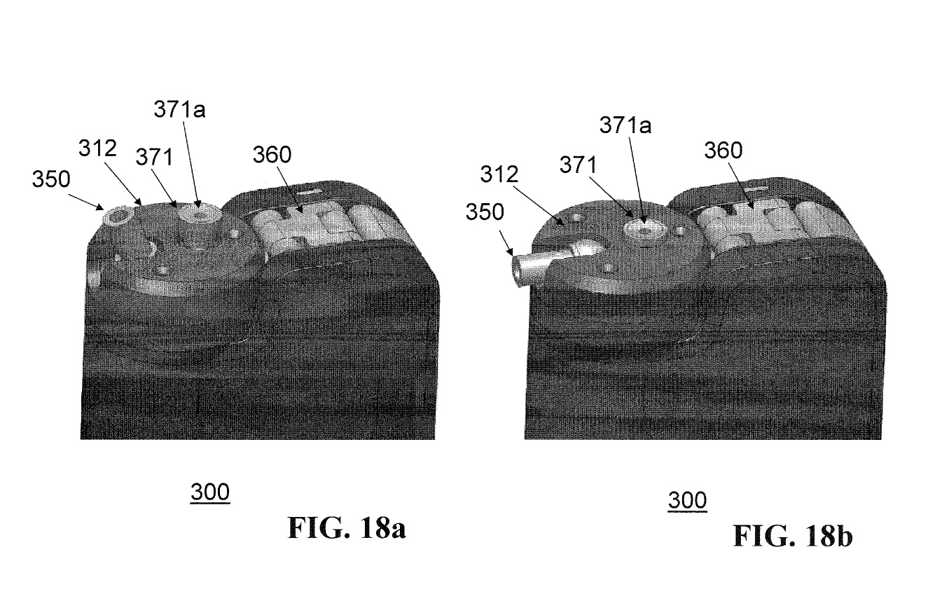

FIGS. 18a and 18b illustrate a damper knob 371 that is positioned within an opening in the cap 312. The damper knob 371 contacts an intake tube 372 that contacts the damper 370. The damper knob 371 may be moved up and down relative to the cap 312, enabling a user to cause the damper 370 to press on the material within the heating chamber 330. In addition, the cap 312 may be configured to rotate relative to the main body 302. This rotation allows the pipe to be positioned according to user preference. The rotation of the cap 312 also rotates the damper 370, thereby agitating the material within the herb container. For example, the cap 312 may be configured to allow a 360 degree rotation. The damper may further include an agitator 373 having extensions 373a that protrude from the surface of the agitator 373 to enhance the agitation of the material within the heating chamber 330.

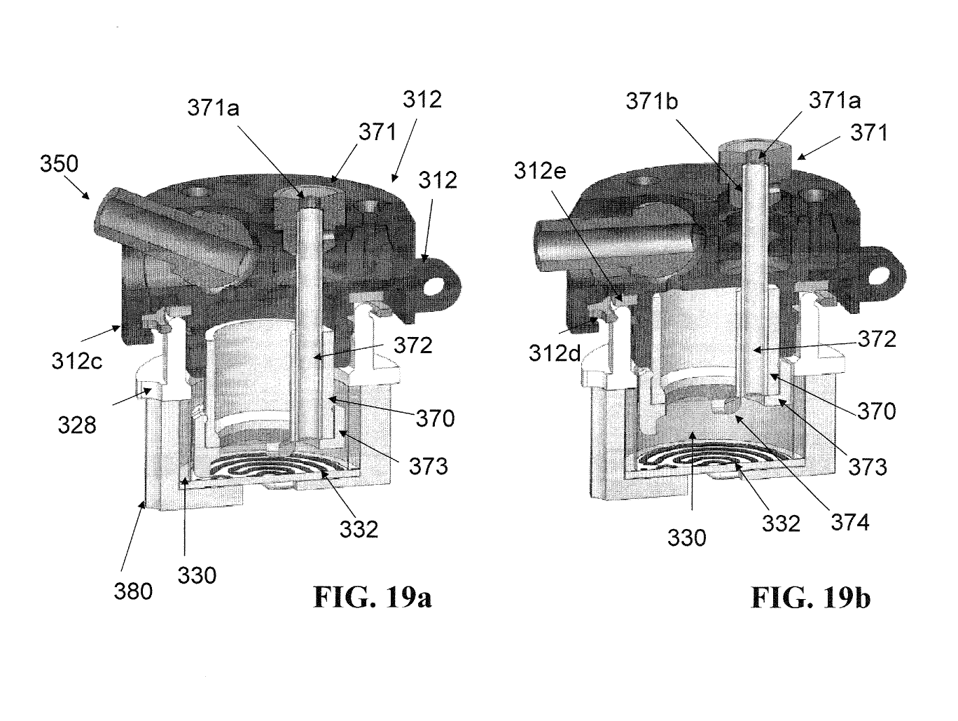

FIGS. 19a and 19b illustrate an assembly of the cap 312 and the heating chamber 330, wherein the cap 312 sits on top of a cap mount 328 that is mounted to an insulation element 380, to be further described herein. These figures illustrate the up and down movement of the damper 370 as the damper knob 371 is moved up or pressed down. As illustrated in the cross section view, the damper knob 371 include an air inlet hole 371a that is in contact with a passageway in the intake tube 372. The damper knob 371 also coupled to the intake tube 372 by an opening 371b, where the intake tube 372 is attached to the damper knob 371 by the intake tube inserted into the opening 371b of the damper knob 371.

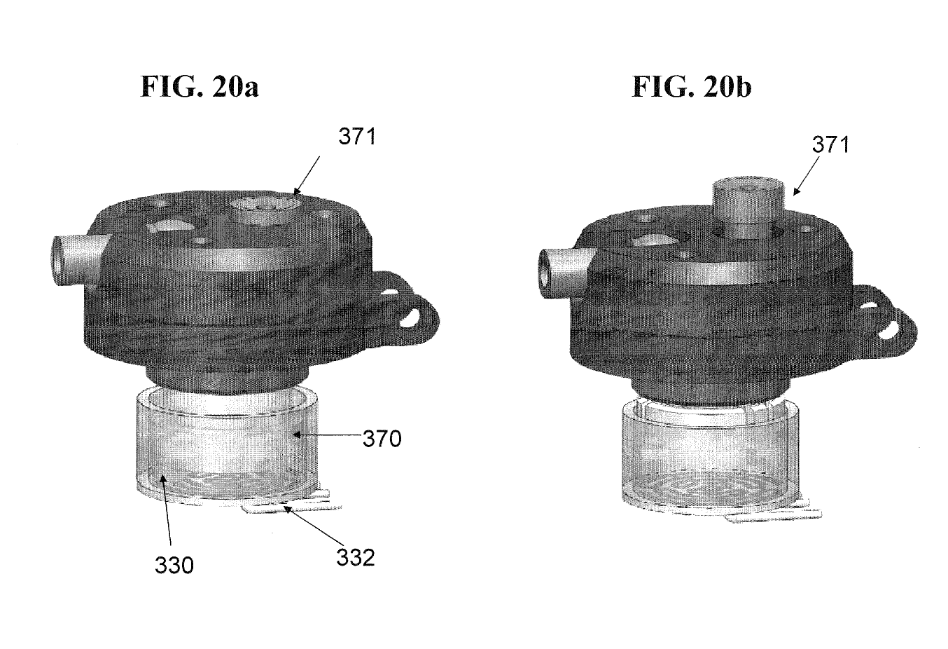

FIGS. 20a and 20b further illustrate the movement of the damper 370 relative to the heating chamber 330, where a user may lift the damper 370 by lifting and dropping the damper knob 371. In addition to the damper 370, the agitator 373 will also be moved by the lifting and dropping of the damper knob 371.

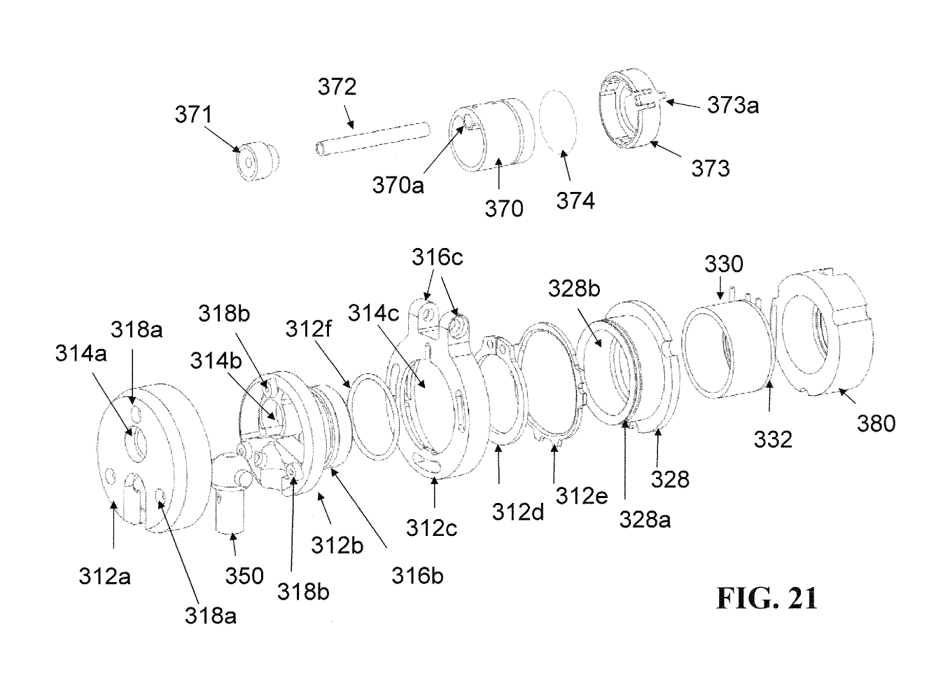

FIG. 21 illustrates a separated view of elements that may be comprised within the vaporizer 300. For example, the damper 370 may further include a screen and/or filter element 374 that prevents herbal material from entering the intake tube 372. In addition, the cap 312 may comprise an outer cap 312a, an inner cap 312b, a cap holder 312c, a c-clip 312d, a snap ring 312e, and an o-ring 312f. The cap 312 is coupled to the cap mount 328 when the vaporizer 300 is in use, as discussed herein, and the insulation element 380 is used to provide insulation to the heater 332. The outer cap 312a and the inner cap 312b includes an outer cap opening 314a and an inner cap opening 314b, respectively, through which the intake tube 372 passes to be attached to the damper 370. The intake tube 372 is attached via a hole 370a in the damper 370. The outer cap 312a and the inner cap 312b are coupled together through the use of a plurality of screws (not shown) inserted into outer cap holes 318a and inner cap holes 318b, respectively.

Thus, an assembly consisting of the outer cap 312a, the pipe mount 350, the inner cap 312b, the damper knob 371, the intake tube 372, the damper 370, the screen filter 374, and the agitator 373 all rotate within the cap mount 328 in the heating chamber 330. The assembly is retained in the cap holder 312c, whereby a portion of the inner cap 312b is inserted through an opening 314c in the cap holder 312c and secured to the cap holder 312c by a c-clip 312d, allowing the assembly to rotate with respect to the cap holder 312c. The snap ring 312e is held by tabs on the cap holder 312c, and snaps onto a perimeter groove 328a on an outer portion of the cap mount 328. In one embodiment, the snap ring 312e may be constructed of a pliable material such as a plastic or other polymer. The snap ring 312e provides a mechanical force to hold the cap 312 onto the cap mount 328. The o-ring 312f provides a seal between an inside portion 328b of the cap mount 328. The o-ring 312f may be constructed of plastic, silicone, or any other pliable material suitable to provide the sealing purposes for which it is intended. The cap holder 312c attaches to the main body 302 through the articulated hinge 360 attached a joint 316c of the cap holder 312.

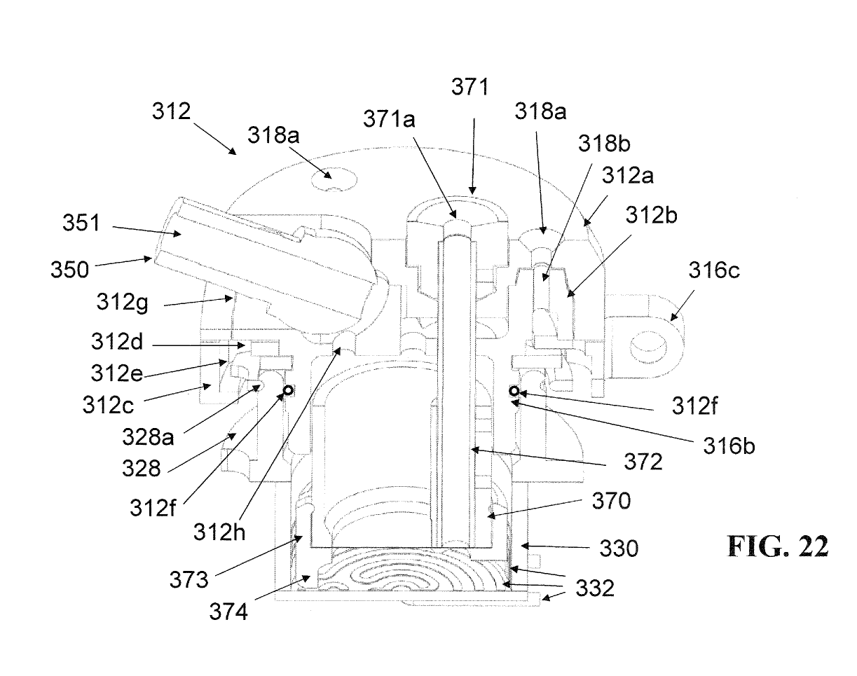

FIG. 22 illustrates an example of the air flow through the vaporizer. Air is drawn through an opening 371a in the damper knob 371 and travels through the intake tube 372 and the damper 370 to the heating chamber 330. The herb is vaporized by the heating element 332, and the vaporized air exits through an opening 351 in the pipe mount 350 and an attached mouthpiece (not illustrated). The damper 370 includes the hole 370a, which is an opening configured to receive the intake tube 372, and the damper knob 371 also includes an opening 371b configured to receive the intake tube 372. Thus, the damping mechanism includes an air channel that extends through the cap 312 into the heating chamber 330. The hole 370a in the damper 370 thus provides an air channel that communicates with a bottom portion of the cap 312. The bottom portion of the cap 312 includes an opening 312g configured to receive the pipe mount 350. The bottom portion of the inner cap 312b may also be configured with a smaller opening 312h that communicates with an opening 351 in the pipe mount 350. As discussed, the o-ring 312f may be used to provide the seal between the inner cap 312b and the cap mount 328.

A one-way valve may be provided in a mouthpiece or the pipe mount 350, or in the cap 312, such as in the damper knob 371 or intake tube 372. The one-way valve may function to trap vapors in the cap 312 unless suction is provided to the mouthpiece, thereby only allowing the vapors to escape through the mouthpiece

The pipe mount 350 may also be configured to communicate with the smaller opening 312h in the bottom portion of the cap 312 only when the pipe mount 350 is rotated to a particular position. This smaller opening 312h thereby enables the opening 351 in the mouthpiece extension piece to be closed off from communication with the heating chamber 330 by rotating the pipe mount 350 to a second position.

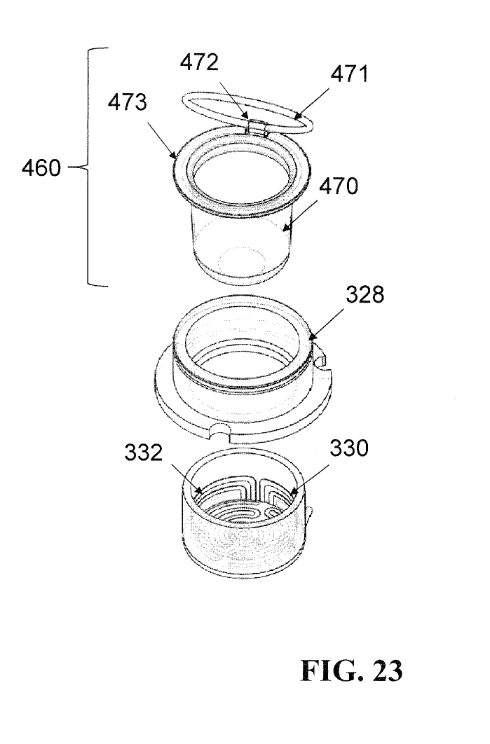

FIG. 23 illustrates an exploded perspective view of a cap mount 328, a heating chamber 330, and a heater 332 configured in accordance with another embodiment of the present invention for use with a vaporizer such as the portable vaporizer 300, where a basket assembly 460 includes a basket 470 that is configured to hold the herbs instead of having the herbs being placed directly into the heating chamber 330. The heating chamber 330 include a heater 332 used to heat the herbs placed into the basket 470. As illustrated, the heater 332 includes heating elements that line the interior wall of the heating chamber 330 in addition to the bottom of the heating chamber 330. The heating elements may more efficiently heat the herb because the heating elements heat both the side and the bottom of the basket 470.

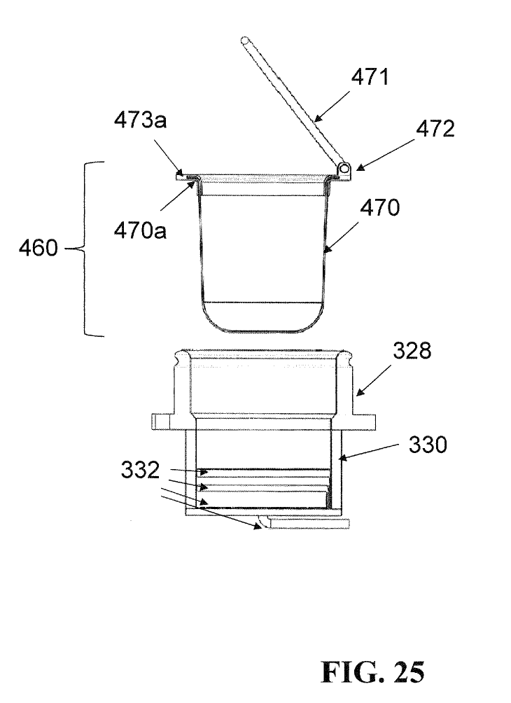

The basket assembly 460 also includes a ringed lip 473. Also referring to FIG. 25, the basket 470 includes a basket lip 470a that is captured within a basket lip capture portion 473a of the ringed lip 473. The ringed lip 473 includes a handle 471 coupled to the ringed lip 473 on a hinge 472. In one example construction, the basket 470 may be constructed of a metal mesh, with the ringed lip 473, the hinge 472, and the handle 471 being constructed of metal. The metal mesh construction may allow more air to pass through the herb as it is being heated. However, the examples of the materials given should not be considered limiting. For example, the basket 470 may also be constructed of glass, or any material that allows heating of the herbs contained therein through the use of heating element in the heater 332. Thus, any suitable composites, polymers or alloys may be used.

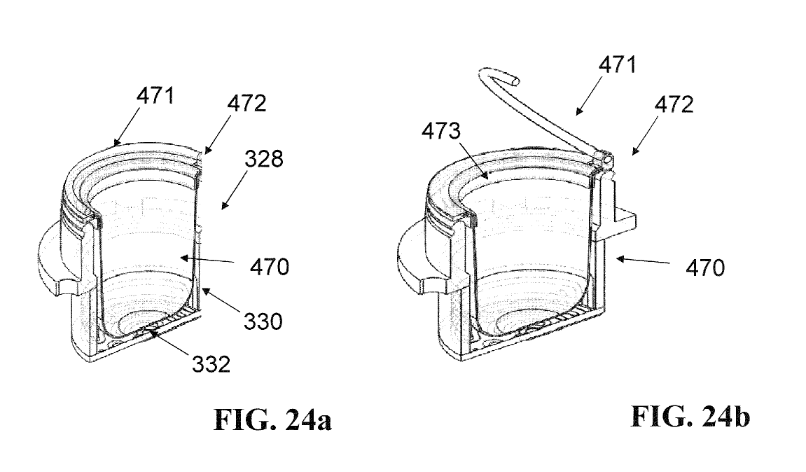

FIGS. 24a and 24b illustrate a perspective cross section of the cap mount 328, the heating chamber 330, and the heater 332, where the basket assembly 460 has been placed into the heating chamber 330. Thus, the basket 470 sits in the heating chamber 330 and is supported by the ringed lip 473 supported on the cap mount 328. The basket assembly 460 may be lifted out of the heating chamber 330 using the handle 471 mounted to the ringed lip 473 by articulating the handle 471 around the hinge 472. In this configuration, the basket assembly 460 is an easily removable container allowing for cleaning any residue in the vaporizer after any herbs or tobaccos are vaporized. The basket 470 of the basket assembly 460 allows the herbs to sit above the heater 332 and provides a space 482 underneath the herb for heated air, i.e., air heated by the heater 332, to gather and be drawn through the basket 470 and up to the pipe mount 350.

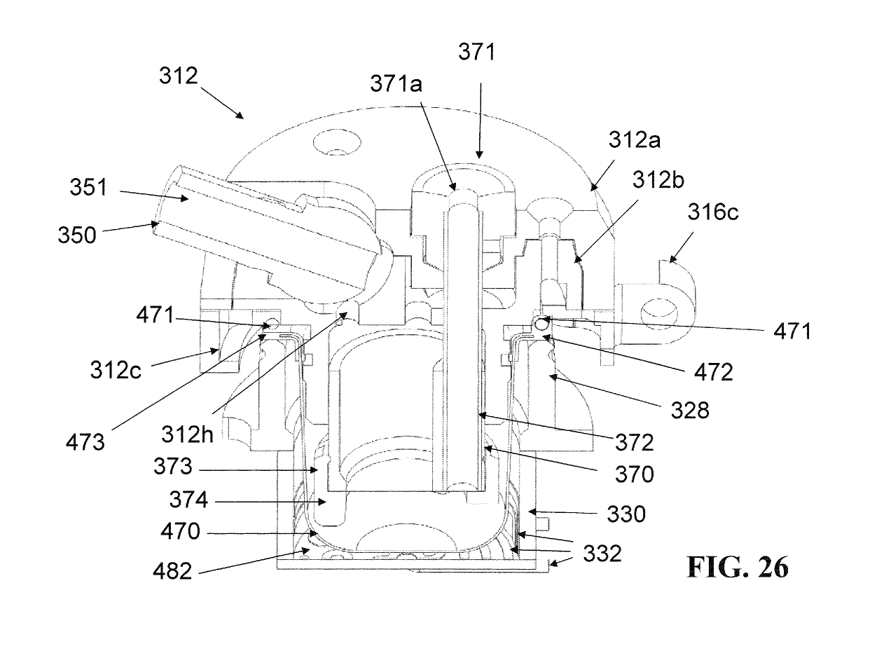

FIG. 26 illustrates the basket assembly 460 inserted into the cap mount 328 and the heating chamber 330, whereby the cap 312 has been snapped onto the cap mount 328 using the snap ring 312e. The other numbered components functions as described with the description contained previously unless otherwise noted herein. For example, an air passageway may consist of the hole 371a in the damper knob 371, the intake tube 372, the damper 370 (through the screen filter 374), the agitator 373, and into the basket 470, and out through the smaller opening 312h, and the pipe mount 350 through a mouthpiece attached to the pipe mount 350. A one-way valve may be incorporated anywhere in the air passageway to prevent or minimize an exit of vapors other than the pipe mount 350. Further, for purposes of reducing clutter, certain elements such as the c-clip 312d, the snap ring 312e, and the o-ring 312f have not been reproduced in the figure. However, those skilled in the art will understand they may be integrated the same way to function with the approach offered by the use of the basket assembly 460. The basket 470 of the basket assembly 460 is supported by the ringed lip 473 sitting on the cap mount 328. Also illustrated is the handle 471 sitting flush on top of the ringed lip 473.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

What is claimed is:

1. A portable vaporizer comprising:
a main body;
a heating assembly coupled to the main body;
a removable basket in thermal communication with the heating assembly and configured to allow an herb placed in the removable basket to be vaporized by the heating assembly; and
an agitator device configured to agitate the herb placed in the removable basket to achieve a more uniform vaporization of the herb.

2. The portable vaporizer according to claim 1, wherein the removable basket comprises a handle for retrieving the removable basket from the heating assembly.

3. The portable vaporizer according to claim 1, further comprising a mouthpiece configured to be removably attach to the main body to be in air flow communication with the heating assembly, and wherein the main body further comprises an opening configured to receive and store the mouthpiece when the mouthpiece is not in use.

4. The portable vaporizer according to claim 1, wherein the heating assembly comprises:
a chamber comprising an opening in which the removable basket sits;
a heating element thermally coupled to the chamber; and
a glass layer positioned between the heating element and the chamber.

5. The portable vaporizer according to claim 4, wherein the heating element is a ceramic heating element.

6. The portable vaporizer according to claim 4, further comprising:
a cover configured to provide a seal for the opening of the chamber, the cover comprising an air inlet; and
a one-way valve between the cover and the opening, wherein the one-way valve allows air to be pulled through the air inlet and prevents air from escaping therefrom.

7. The portable vaporizer according to claim 4, wherein the heating assembly further comprises:
a cover configured to provide a seal for the opening of the chamber; and
an multi-linked joint for attaching the cover to the main body.

8. The portable vaporizer according to claim 1, wherein the heating assembly comprises:
a removable heating attachment configured to be removably attached to the main body, wherein the main body is configured to attach interchangeably to the removable heating attachment and another removable heating attachment.

9. The portable vaporizer according to claim 3, wherein the main body comprises:
a battery; and
a power connection configured to mate interchangeably with the removable heating attachment and the other removable heating attachment.

10. The portable vaporizer according to claim 3, wherein the heating assembly comprises a removable cigarette attachment configured to vaporize the contents of a cigarette.

11. The portable vaporizer according to claim 10, wherein the removable cigarette attachment comprises:
a first piece including a tobacco releasing mechanism; and
a second piece insertable into the first piece, the second piece including a cigarette holder configured to receive and hold a cigarette, wherein the tobacco releasing mechanism is positioned in the first piece to release tobacco from the cigarette as the second piece is inserted into the first piece.

12. The portable vaporizer according to claim 11, wherein the tobacco releasing mechanism is a blade that is positioned to slice the cigarette as the second piece is inserted into the first piece.

13. The portable vaporizer according to claim 10, wherein the heating assembly comprises a heating element that extends along the length of the cigarette.

14. The portable vaporizer according to claim 10, wherein the cigarette attachment includes at least one gasket sealing a vaporization chamber of the cigarette attachment.

15. The portable vaporizer according to claim 14, wherein the cigarette attachment further comprises an opening configured to allow air to be drawn through a filter end of the cigarette into the vaporization chamber.
